(19) United States
(12) Patent Application Publication
(10) Pub. No.: US 2011/0208289 A1
(43) Pub. Date: Aug. 25, 2011

(54) Flexible Stent-Grafts

(75) Inventor: Alon SHALEV, Ra'anana (IL)

(73) Assignee: ENDOSPAN LTD., Herzilyia Pituach (IL)

(21) Appl. No.: 13/031,871

(22) Filed: Feb. 22, 2011

Related U.S. Application Data
(60) Provisional application No. 61/307,869, filed on Feb. 25, 2010.

(51) Int. Cl. A61F 2/82

(52) U.S. Cl. 623/1.15; 623/1.35

(57) ABSTRACT

A stent-graft includes a graft and annular stent springs, including first, second, and third tapered stent springs, coupled to a generally tubular portion of the graft. Each of the tapered springs include stent cells that circumferentially taper to a set of one or more circumferentially-adjacent narrowest stent cells within the spring. The first and second tapered springs axially adjacent; the second and third tapered stent springs are axially adjacent. The narrowest stent cell sets of the first and second springs are rotationally positioned on the portion of the graft with a non-zero relative angle shift therebetween, as are the narrowest stent cell sets of the second and third springs.

### CROSS-REFERENCE TO RELATED APPLICATIONS

The present patent application claims priority from U.S. Provisional Application 61/307,869, filed Feb. 25, 2010, entitled, “Stent graft for placement in curved and in fenestrated body lumen,” which is assigned to the assignee of the present application and incorporated herein by reference.

### FIELD OF THE APPLICATION

This present application relates generally to prostheses, and specifically to tubular prostheses, including endovascular grafts and stent-grafts.

### BACKGROUND OF THE APPLICATION

Endovascular prostheses are sometimes used to treat aortic aneurysms. Such treatment includes implanting a stent or stent-graft within the diseased vessel to bypass the anomaly. An aneurysm is a sac formed by the dilation of the wall of the artery. Aneurysms may be congenital, but are usually caused by disease or, occasionally, by trauma. Aortic aneurysms which commonly form between the renal arteries and the iliac arteries are referred to as abdominal aortic aneurysms (“AAAs”). Other aneurysms occur in the aorta, such as thoracic aortic aneurysms (“TAAs”) and aortic uni-iliac (“AUI”) aneurysms.

A conventional stent-graft typically includes a radially-expandable stent, formed from a plurality of uniform annular stent springs, and a cylindrically-shaped graft material to which the stent springs are coupled. Stent grafts are known for use in reinforcing or holding open the interior wall of body lumens, such as blood vessels.

PCT Publication WO 2008/107885 to Shalev et al., and US Patent Application Publication 2010/0063575 to Shalev et al. in the US national stage thereof, which are incorporated herein by reference, describe a multiple-component expandable endoluminal system for treating a lesion at a bifurcation, including a self expandable tubular root member having a side-looking engagement aperture, and a self expandable tubular trunk member comprising a substantially blood impervious polymeric liner secured therealong. Both have a radially-compressed state adapted for percutaneous intraluminal delivery and a radially-expanded state adapted for endoluminal support.

The following references may be of interest:

U.S. Pat. No. 4,938,740 to Melbin

U.S. Pat. No. 5,824,040 to Cox et al.

U.S. Pat. No. 7,044,962 to Elliott

U.S. Pat. No. 7,279,003 to Berra et al.

U.S. Pat. No. 7,544,160 to Gross

US Patent Application Publication 2006/0229709 to Morris et al.

US Patent Application Publication 2006/0241740 to Vardi et al.

US Patent Application Publication 2008/0109066 to Quinn

US Patent Application Publication 2010/0063575 to Shalev et al.

PCT Publication WO 09/118,733 to Karasik

PCT Publication WO 10/031,060 to Tuval et al.

### SUMMARY OF APPLICATIONS

Some embodiments of the present invention provide an endovascular stent-graft, which comprises a stent and a graft. The stent comprises a plurality of annular stent springs, which include at least first, second, and third tapered stent springs, which are coupled to a tubular portion of the graft. The first and second tapered stent springs are axially adjacent, and the second and third tapered stent springs are axially adjacent. Each of the first, second, and third tapered stent springs comprises stent cells that vary in size, and taper to a set of one or more circumferentially-adjacent smallest stent cells within the spring, such that the tapered stent springs comprise respective smallest stent cell sets. The smallest stent cell sets of the first and second tapered stent springs are rotationally positioned on the graft with a first relative non-zero angle shift (i.e., rotational offset) between the respective circumferential centers of the smallest stent cell sets, and the smallest stent cell sets of the second and third tapered stent springs are rotationally positioned on the graft with a second relative non-zero angle shift between the respective circumferential centers of the smallest stent cell sets. The first and second relative angle shifts may different from or equal to each other. Typically, the smallest stent cells within the spring are those stent cells within the spring that have the smallest lateral width, measured longitudinally in a direction parallel to a longitudinal axis of the graft.

This arrangement of the tapered stent springs provides the stent-graft with greater flexibility than would be obtained if the stent-springs were not tapered. At the same time, this arrangement provides greater axial support for the stent-graft than would be obtained if the stent-springs were not tapered and simply comprised small stent cells. The stent-graft is able to conform to a tortuous body lumen without kinking, even if the body lumen has a complex curved shape containing compound curves. The stent-graft does not need to be customized for the particular geometry of a given body lumen, because the arrangement of the tapered stent springs allows the stent-graft to bend in all directions, while only partially compromising the axial support and collapse-resistance of the stent-graft.

For some applications, at least a portion of the stent cells are closed, i.e., form a structure with a continuous (uninterrupted) perimeter, e.g., are diamond-shaped. Alternatively or additionally, at least a portion of the stent cells are open, i.e., form a structure having a broken, incomplete perimeter, e.g., have a serpentine shape.

For some applications, a portion of the stent cells of a tapered stent spring are open, and one of the stent cells is closed. The graft may be shaped so as to define a side-facing fenestration surrounded by the closed stent cell. The closed cell may coincide with a border of the fenestration, or may be slightly recessed from the border. Typically, the closed stent cell is at least as large as the other stent cells in its tapered stent spring.

For some applications, the stent-graft is used to treat an aneurysm, such as an aortic aneurism, or an aneurism of another blood vessel. For example, the aneurism may be of the sub-renal aorta.

There is therefore provided, in accordance with an application of the present invention, apparatus including a stent-graft, which includes:

a graft, which is shaped so as to define at least one generally tubular portion having a longitudinal axis, when the stent-graft is in a radially-expanded state; and

annular stent springs, which include at least first, second, and third tapered stent springs, which tapered stent springs are coupled to the portion of the graft, and each of which tapered stent springs includes stent cells that taper to a set of one or more circumferentially-adjacent narrowest stent cells within the spring, such that the tapered stent springs include respective narrowest stent cell sets, when the stent-graft is in its radially-expanded state, wherein the narrowest stent cells within the spring are those stent cells within the spring that have the smallest lateral width, measured longitudinally in a direction parallel to the longitudinal axis,

wherein the first and the second tapered stent springs are axially adjacent, and the second and the third tapered stent springs are axially adjacent, and

wherein the narrowest stent cell sets of the first and the second tapered stent springs are rotationally positioned on the portion of the graft with a first non-zero relative angle shift between respective circumferential centers of the narrowest stent cell sets thereof, and the narrowest stent cell sets of the second and the third tapered stent springs are rotationally positioned on the portion of the graft with a second non-zero relative angle shift between respective circumferential centers of the narrowest stent cell sets thereof.

For some applications, the first and the second relative angle shifts are equal; alternatively, they are not equal. For some applications, the first and the second tapered stent springs do not touch one another when the stent-graft is straight in its radially-expanded state. For some applications, each of the narrowest stent cell sets includes exactly one of the stent cells.

For some applications, each of the first and the second relative angle shifts is between 120 and 150 degrees. Alternatively, for some applications, each of the first and the second relative angle shifts is between 30 and 120 degrees. For some applications, each of the first and the second relative angle shifts is less than 90 degrees.

Typically, each of the first and the second relative angle shifts is greater than 5 degrees.

For some applications, the portion of the graft is disposed inside the tapered stent springs. Alternatively, the portion of the graft is disposed outside the tapered stent springs.

For some applications, at least a portion of the stent cells are closed. For some applications, at least a portion of the stent cells are diamond-shaped.

For some applications, at least a portion of the stent cells are open. For some applications, at least a portion of the stent cells are serpentine-shaped. For some applications, at least a portion of the stent cells are zigzagged.

For some applications, a portion of the stent cells of one of the tapered stent springs are open, and one of the stent cells of the one of the tapered stent springs is closed. For some applications, the portion of the graft is shaped so as to define a side-facing fenestration surrounded by the closed one of the stent cells. For some applications, the closed one of the stent cells is at least as large as the other stent cells of the one of the tapered stent springs.

For some applications, the stent-graft includes a bifurcated portion.

For some applications, the graft includes a polymer.

For some applications, the polymer is selected from the group consisting of: a fluoropolymer, polytetrafluoroethylene, a polyester, polyethylene, and polyethylene terephthalate.

For some applications, the stent springs include a superelastic alloy. For some applications, the stent springs include a material selected the group consisting of: stainless steel, a cobalt chromium alloy, a platinum/tungsten alloy, and a nickel-titanium alloy.

There is further provided, in accordance with an application of the present invention, apparatus including a stent-graft, which includes:

a graft, which is shaped so as to define at least one generally tubular portion when the stent-graft is in a radially-expanded state; and

annular stent springs, which include at least first, second, and third tapered stent springs, which tapered stent springs are coupled to the portion of the graft, and each of which tapered stent springs includes stent cells that taper to a set of one or more circumferentially-adjacent smallest stent cells within the spring, such that the tapered stent springs include respective smallest stent cell sets, when the stent-graft is in its radially-expanded state,

wherein the first and the second tapered stent springs are axially adjacent, and the second and the third tapered stent springs are axially adjacent,

wherein the smallest stent cell sets of the first and the second tapered stent springs are rotationally positioned on the portion of the graft with a first non-zero relative angle shift between respective circumferential centers of the smallest stent cell sets thereof, and the smallest stent cell sets of the second and the third tapered stent springs are rotationally positioned on the portion of the graft with a second non-zero relative angle shift between respective circumferential centers of the smallest stent cell sets thereof, and

wherein each of the first and the second relative angle shifts is greater than 5 degrees.

For some applications, the first and the second relative angle shifts are equal; alternatively, they are not equal. For some applications, each of the smallest stent cell sets includes exactly one of the stent cells. For some applications, at least a portion of the stent cells are selected from the group consisting of: closed stent cells, and open stent cells.

The present invention will be more fully understood from the following detailed description of embodiments thereof, taken together with the drawings, in which:

### BRIEF DESCRIPTION OF THE DRAWINGS

FIG. 1 is a schematic illustration of an endovascular stent-graft, in accordance with an application of the present invention;

FIGS. 2A-B are schematic illustrations of arrangements of annular stent springs of the stent-graft of FIG. 1, in accordance with respective applications of the present invention;

FIG. 3 is a schematic illustration of a single tapered stent spring of the stent-graft of FIG. 1, in accordance with an application of the present invention;

FIGS. 4A-B are schematic illustrations of a single tapered stent spring of the stent-graft of FIG. 1, in accordance with respective applications of the present invention;

FIG. 5 is a schematic illustration of the stent-graft of FIG. 1 deployed in a body lumen of a subject, in accordance with an application of the present invention;

FIG. 6 is a schematic illustration of a bifurcated configuration of the stent-graft of FIG. 1, in accordance with an application of the present invention;

FIGS. 7A-B are schematic illustrations of an exemplary deployment of the stent-graft of FIG. 1 in iliac arteries, in accordance with an application of the present invention; and

FIG. 8 is a schematic illustration of an arrangement of annular stent springs of the iliac stent-graft of FIGS. 7A-B, in accordance with an application of the present invention.

### DETAILED DESCRIPTION OF APPLICATIONS

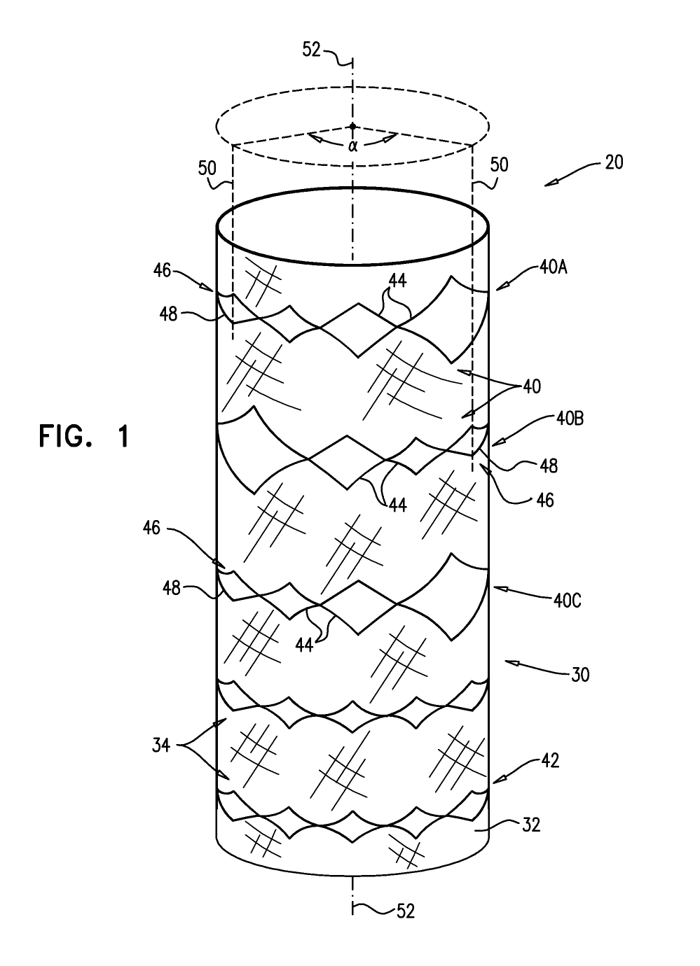

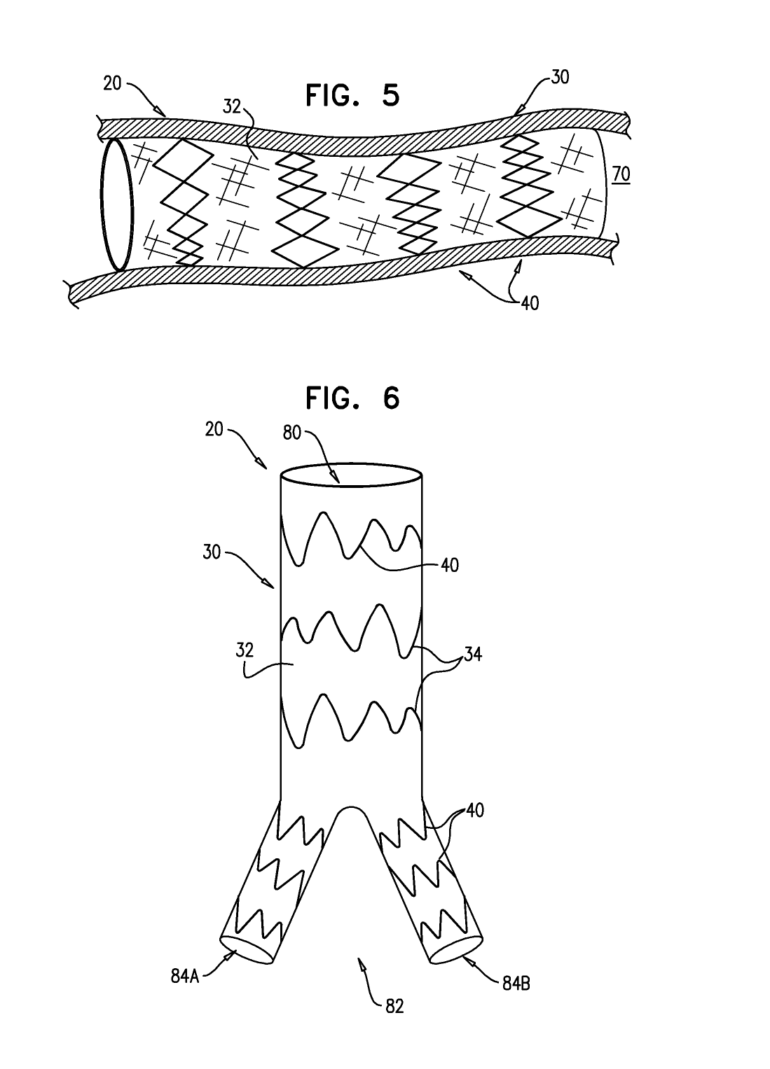

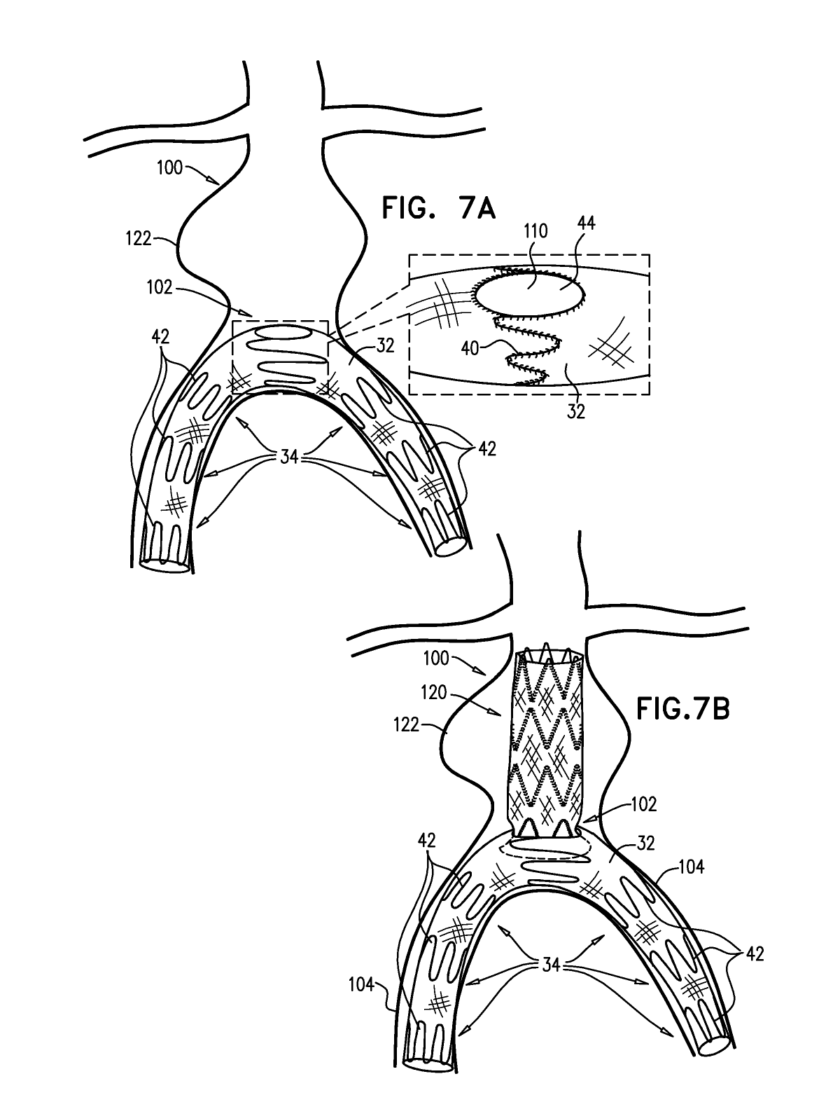

FIG. 1 is a schematic illustration of an endovascular stent-graft 20, in accordance with an application of the present invention. Stent-graft 20 is typically configured to be implanted in at least one blood vessel (such as an artery) in a vicinity of an aneurysm, such as described hereinbelow with reference to FIGS. 5 and 7A-B. Stent-graft 20 comprises a stent 30 (which serves as a structural member) and a graft 32, at least a portion of which is generally tubular and is shaped as a fluid flow guide. In the exemplary configuration shown in FIG. 1, the entire graft is generally tubular. Typically, when stent-graft 20 is in a radially-expanded state, at least portions of the stent and the graft are generally tubular. Stent 20 comprises a plurality of annular stent springs 34, which are coupled to graft 32.

For some applications, stent springs 34 comprise a metal, such as stainless steel, a cobalt chromium alloy, a platinum/tungsten alloy, or a nickel-titanium alloy. Alternatively or additionally, the stent springs comprise a self-expanding elastic material, such as a superelastic alloy. Alternatively or additionally, the stent springs comprise a superelastic alloy, such as Nitinol. For some applications, stent 30 comprises a wire stent, while for other applications, stent 30 comprises a ribbon stent. For some applications, stent 30 is formed from tubing.

Graft 32 comprises at least one biologically-compatible substantially fluid-impervious flexible sheet, which is coupled to stent 30, either outside or within the stent, such as by stitching (e.g., sutures), and covers either an external or an internal surface of at least a portion of the stent. In other words, graft 32 may be disposed inside or outside of stent springs 34. The flexible sheet may comprise, for example, a polymer (e.g., a fluoropolymer, such as polytetrafluoroethylene, or a polyester, such as polyethylene or polyethylene terephthalate (PET)), natural tissue (e.g., saphenous vein or collagen), or a combination thereof.

For some applications, stent-graft 20 is configured to initially be positioned in a delivery catheter in a radially-compressed state, and to assume its radially-expanded state upon being deployed from the delivery catheter. When in the radially-expanded state, stent-graft 20 is shaped so as to define a lumen. FIGS. 1, 5, 6, and 7A-B show the stent-graft in the radially-expanded state. For some applications, the stent-graft is heat-set to assume the radially-expanded state. For some applications, when in its radially-expanded state, an axial length of stent-graft 20 is at least 3 cm, no more than 30 cm, and/or between 3 and 30 cm.

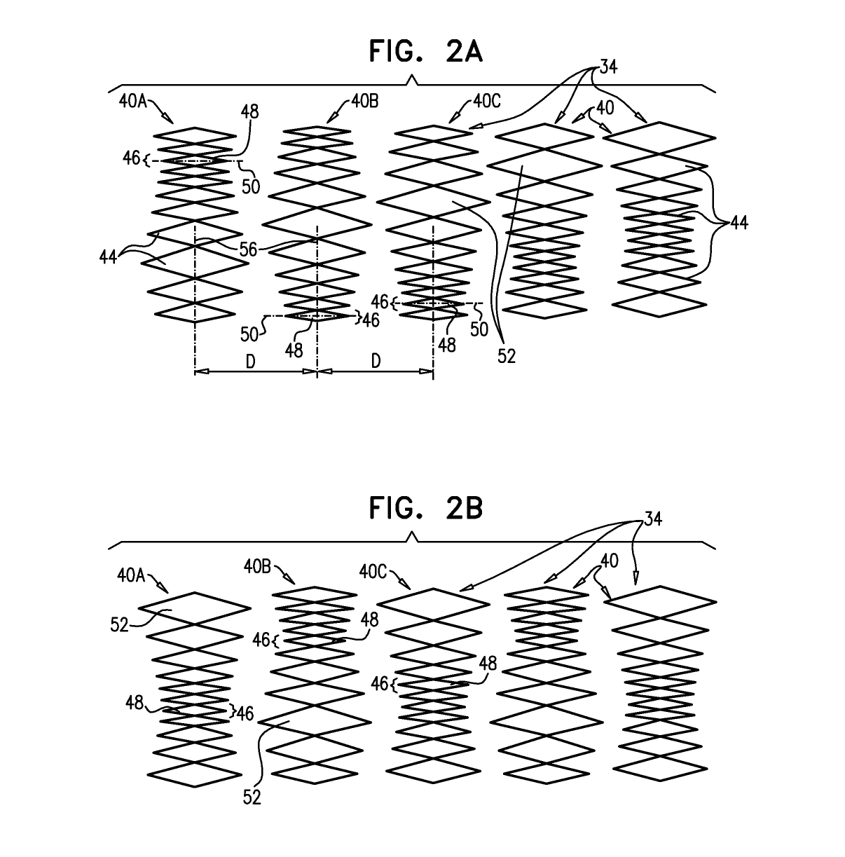

Reference is still made to FIG. 1, and is now also made to FIGS. 2A-B, which are schematic illustrations of arrangements of annular stent springs 34, in accordance with respective applications of the present invention. Although each of annular stent springs 34 typically defines a complete loop, for clarity of illustration FIG. 1 shows only a portion of each of the annular stent springs (about half of each spring); the remainder of each spring is on the opposite side of stent-graft 20, hidden by graft 32. FIGS. 2A-B, 3, and 4A-B show annular stent springs 34 opened and flattened. In actual practice, the top and bottom ends of each of the springs are typically connected with each other, to form a complete ring.

Annular stent springs 34 include one or more tapered stent springs 40, typically at least three, such as at least five tapered stent springs 34. Optionally, annular stent springs 34 further include one or more non-tapered springs 42. For example, tapered stent springs 40 may include at least first, second, and third tapered stent springs 40A, 40B, and 40C, which are coupled to the tubular portion of graft 32. First and second tapered stent springs 40A and 40B are axially adjacent, and second and third tapered stent springs 40B and 40C are axially adjacent, along a longitudinal axis 43 of stent-graft 20. As used in the present application, including in the claims, two stent springs are “axially adjacent” each other when they are longitudinally next to each other, without any other stent springs longitudinally intervening between the two stent springs (although at least partially axially-oriented interconnecting members that are adapted to connect between axially-adjacent stent springs may optionally intervene).

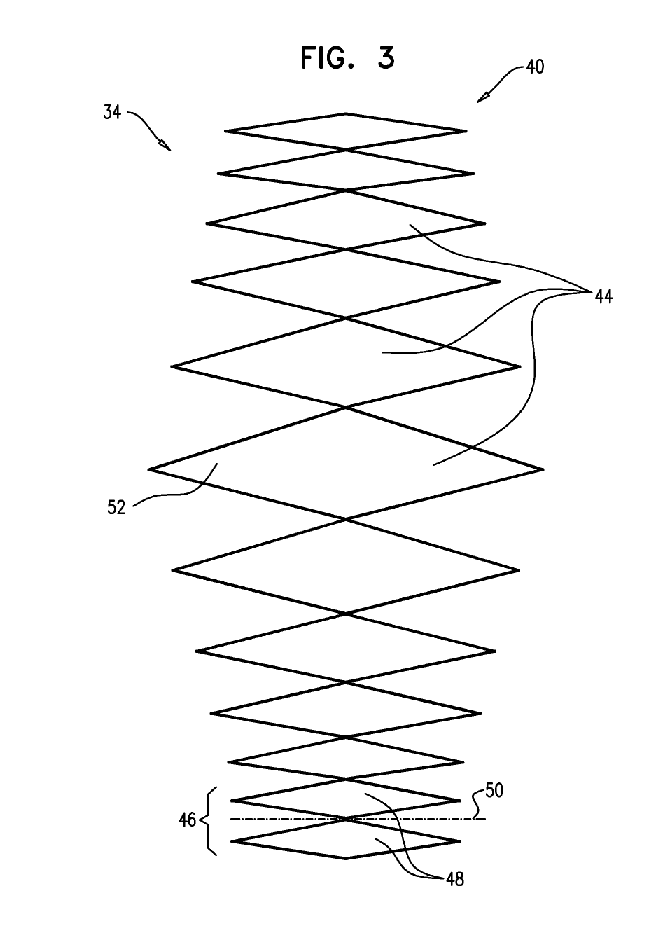

At least each of tapered stent springs 40A, 40B, and 40C comprises stent cells 44 that vary in size, and circumferentially taper (around a circumference of the stent spring) to a set 46 of one or more circumferentially-adjacent smallest stent cells 48 within the spring, such that the tapered stent springs comprise respective smallest stent cell sets 46, when stent-graft 20 is in its radially-expanded state. (More than three tapered stent springs may optionally be provided, optionally adjacent to one another.) For some applications, each of smallest stent cell sets 46 comprises exactly one smallest stent cell 48, such as shown in FIGS. 1, 2A-B, 4A-B, 5, 6, 7A-B, and 8. For other applications, one or more of smallest stent cell sets 46 comprises a plurality of smallest stent cells 48, such as two or three, which have the same small size, such as described hereinbelow with reference to FIG. 3. For some applications, one or more of the smallest stent cell sets comprise exactly one smallest stent cell 48, and one or more of the smallest stent cell sets comprise more than one smallest stent cell 48. In any case, smallest stent cell sets 46 have respective circumferential centers 50, such as indicated in FIGS. 1, 2A, and 3. Although cells 44 are shown having similar shapes, they may alternatively have different shapes from one another. Each of tapered stent springs 40 also defines one or more largest stent cells 52. Each of tapered stent springs 40 typically is only one stent cell wide (along longitudinal axis 52).

Smallest stent cell sets 46 of first and second tapered stent springs 40A and 40B are rotationally positioned on graft 32 with a first relative non-zero angle α (alpha) shift (i.e., rotational offset) between respective circumferential centers 50 of smallest stent cell sets 46, and smallest stent cell sets 46 of second and third tapered stent springs 40B and 40C are rotationally positioned on graft 32 with a second relative non-zero angle shift between respective circumferential centers 50 of smallest stent cell sets 46. The first and second angle shifts are measured with respect to a longitudinal axis 54 of stent-graft 20. For some applications, the first and second relative angle shifts are different from each other, while for other applications, the first and second relative angle shifts are equal. For example each of the first and second relative angle shifts may be (a) at least 5 degrees, such as at least 10 degrees, (b) at least 150 degrees, no more than 210 degrees, and/or between 150 and 210 degrees, such as 180 degrees (as shown in FIG. 2B), (c) at least 30 degrees, no more than 120 degrees, and/or between 30 and 120 degrees, such as 72 degrees (as shown in FIG. 2A), 90 degrees, (d) less than 90 degrees, or (e) at least 120 degrees, no more than 150 degrees, and/or between 120 and 150 degrees. Largest stent cells 54 of adjacent tapered stent springs are also typically rotationally shifted (i.e., offset) by the same angles as smallest stent cells 48. For applications in which each of sets 46 includes exactly one smallest stent cell 48, the smallest stent cells of adjacent tapered stent springs may be rotationally shifted (i.e., offset) by the angles described above.

For some applications, longitudinally-adjacent stent springs 34 (such as first and second tapered stent springs 40A and 40B, and/or second and third tapered stent springs 40B and 40C) do not touch one another when the stent-graft is in a radially-expanded, straight state, such as shown in FIGS. 1, 2A-B, 5, 6, 7A-B, and 8. These stent springs are thus independent form another, and are longitudinally spaced along graft 32, with respective spaces between each pair of stent springs. For example, the stent springs may be spaced apart from one another by a spacing distance D (labeled in FIG. 2A). Spacing distance D may be measured between respective longitudinal centers 56 of adjacent stent springs, as shown in FIG. 2A. Spacing distance D may vary between different adjacent pairs of adjacent stent springs, or may be the same, as shown in FIG. 2A. It is noted that the stent springs may touch each other, or nearly touch each other except for intervening graft material, when the stent-graft is axially curved, such as when placed in a curved blood vessel, and/or when the stent-graft is initially in a radially-compressed state for delivery.

Alternatively, for some applications, at least some of (e.g., all of) the stent springs 34 are interconnected; either longitudinal adjacent cells of adjacent stent springs may be connected, or separate connecting struts may be provided (configurations not shown).

Reference is now made to FIG. 3, which is a schematic illustration of a single tapered stent spring 40, in accordance with an application of the present invention. In this exemplary configuration, smallest stent cell set 46 of tapered stent spring 40 comprises two smallest stent cells 48. Circumference center 50 of smallest stent cell 46 is located circumferentially between the two smallest stent cells 48. In configurations in which the smallest stent cell set includes an odd number of stent cells (not shown), the circumferential center may coincide with a circumferential center of the middle stent cell.

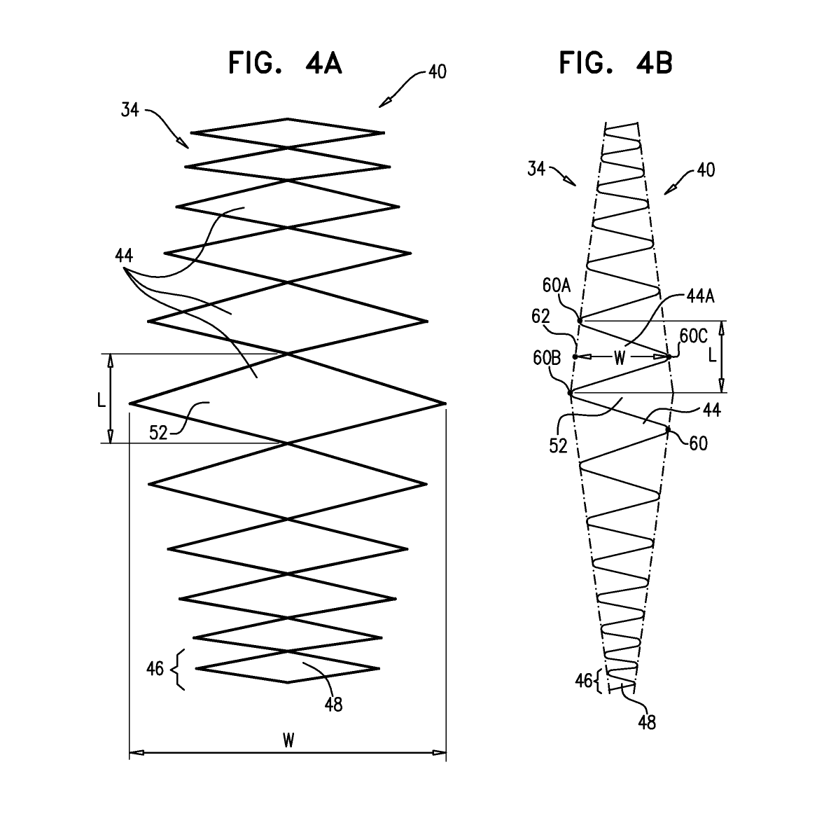

Reference is now made to FIGS. 4A-B, which are schematic illustrations of a single tapered stent spring 40, in accordance with respective applications of the present invention. For some applications, such as shown in FIG. 4A, at least a portion, such as all, of stent cells 44 are closed, i.e., form a structure with a continuous (uninterrupted) perimeter. For example, all or a portion of the stent cells may be diamond-shaped, such as shown in FIG. 4A, ovoid-shaped, or elliptically-shaped (configurations not shown). Each of cells 44 has a circumferential length L, measured along a circumference of the tapered stent spring, and a lateral width W, measured longitudinally in a direction parallel to axis of 54 of stent-graft 20 (FIG. 1). At least a portion of contiguous stent cells 44 vary in size, either in length L, width W, or both length and width W. Cells 44 taper to set 46 of one or more smallest stent cells 48. Typically, smallest stent cells 48 are those cells having the smallest width W (in which case smallest cells 48 are narrowest stent cells 48), and, optionally, the smallest length L. Alternatively, smallest stent cells 48 are those cells having the smallest length L. Further alternatively, smallest stent cells 48 are those cells having the smallest area. In the exemplary configuration shown in FIG. 4A, set 46 includes exactly one smallest stent cell 48. As used in the present application, including in the claims, “taper” means to monotonically decrease in size (i.e., to never increase, such that circumferentially-adjacent cells may be the same size).

For some applications, such as shown in FIG. 4B, at least a portion, such as all, of stent cells 44 are open, i.e., form a structure having a broken, incomplete perimeter. For example, all or a portion of the stent cells may have a serpentine shape, such as shown in FIG. 4B, a triangular shape, or a sinusoid shape (configurations not shown). The serpentine shape may have curved turning points 60, as shown in FIG. 4B, or sharp turning points 60 (i.e., may be zigzagged) (configuration not shown). Each of cells 44 has circumferential length L, measured along a circumference of the tapered stent spring, and a lateral width W, measured longitudinally in a direction parallel to axis of 54 of stent-graft 20 (FIG. 1). For example, assume a cell 44A is extends circumferentially from a turning point 60A to a turning point 60B on the same axially side of the cell, with a turning point 60C on the other side axial side of the cell, circumferentially between points 60A and 60B. Width W of cell 44A may be measured between (a) turning point 60C and (b) a midpoint of a line 62 that connects points 60A and 60B. At least a portion of contiguous stent cells 44 vary in size, either in length L, width W, or both length and width W. Cells 44 taper to set 46 of one or more smallest stent cells 48. In the exemplary configuration shown in FIG. 4B, set 46 includes exactly one smallest stent cell 48.

Reference is still made to FIGS. 4A-B. For some applications, the width W of smallest stent cell(s) 48 equals between 20% and 60% of the average width W of all of cells 44 in tapered stent spring 40, and/or between 10% and 30% of the width W of largest stent cell(s) 52 of tapered stent spring 40. For some applications, the width W of largest stent cell(s) 52 equals between 150% and 500% of the average width W of all of cells 44 in tapered stent spring 40. For some applications, the width W of smallest stent cell(s) 48 equals between 2% and 20% of the circumference of tapered stent spring 40. For some applications, the width W of largest stent cell(s) 52 equals between 10% and 40% of the circumference of tapered stent spring 40. For some applications, the average width W of all of cells 44 in tapered stent spring 40 equals between 6% and 30% of the circumference of tapered stent spring 40.

Reference is now made to FIG. 5, which is a schematic illustration of stent-graft 20 deployed in a body lumen 70 of a subject, in accordance with an application of the present invention. For example, body lumen 70 may be a blood vessel, such as an artery, e.g., an aneurysmatic artery. The body lumen may be tortuous, i.e., may have one or more curved segments. As mentioned above, stent-graft 20 may be configured to initially be positioned in a delivery catheter in a radially-compressed state, and to assume a radially-expanded state upon being deployed from the delivery catheter. Stent-graft 20 may be implanted using techniques for implanting stent-grafts known in the art, and/or described in the patent applications incorporated hereinbelow by reference. Stent-graft 20 is shown in its radially-expanded state.

Reference is now made to FIG. 6, which is a schematic illustration of a bifurcated configuration of stent-graft 20, in accordance with an application of the present invention. Other than as described below, this configuration is generally similar to the configurations described hereinabove with reference to FIGS. 1-5. In this configuration, stent-graft 20 is shaped so as to define a main lumen 80, and a bifurcated downstream end 82, which defines first and second generally tubular downstream lumens 84A and 84B that are in fluid communication with main lumen 80. One or more of the lumens may comprise tapered stent springs 40, arranged such as described hereinabove. Graft 20 is thus shaped so as to define at least one generally tubular portion, i.e., main lumen 80, first downstream lumen 84A, and/or second downstream lumen 84B.

Reference is now made to FIGS. 7A-B, which are schematic illustrations of an exemplary deployment of endovascular stent-graft 20 in iliac arteries 104, in accordance with an application of the present invention. Stent-graft 20 is shown in its radially-expanded state. As shown in FIG. 7A, during a first portion of a deployment procedure, stent-graft 20 is deployed in iliac arteries 104, such that the stent-graft spans both iliac arteries, in a vicinity of a aorto-iliac bifurcation 102 of an abdominal aneurysmatic aorta 100. One of tapered stent springs 40 is positioned at aorto-iliac bifurcation 102. A portion of stent cells 44 of this tapered stent spring are open, and one of stent cells 44 is closed. Typically, graft 32 is shaped so as to define a side-facing fenestration 110 surrounded by the closed one of the stent cells. The closed cell may coincide with a border of the fenestration, or may be slightly recessed from the border. Typically, the closed stent cell is at least as large as the other stent cells of the one of the tapered stent springs; for example, the closed stent cell may be the largest stent cell of the one of the tapered stent springs. Stent-graft 20 is oriented in the iliac arteries such that side-facing fenestration 110 faces aorto-iliac bifurcation 102.

As shown in FIG. 7B, an additional aortic stent-graft 120 is implanted in aorta 100, spanning an abdominal aneurysm 122, from renal arteries 124 to aorto-iliac bifurcation 102. Aortic stent-graft is coupled to stent-graft 20 with a blood-tight interface. For example, an hour-glass shaped region at one end of aortic stent-graft 120 may be disposed within fenestration 110 of stent-graft 20. The hour-glass-shaped region of aortic stent-graft 120 and the border of fenestration 110 are together configured to provide blood-tight interface. Optionally, aortic stent-graft 120 comprises tapered stent springs 40, such as described hereinabove with reference to FIGS. 1-5.

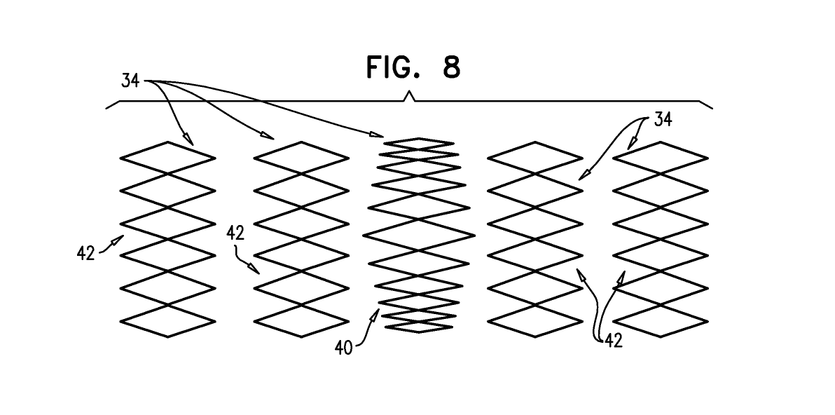

Reference is made to FIG. 8, which is a schematic illustration of an arrangement of annular stent springs 34, in accordance with an application of the present invention. FIG. 8 shows annular stent springs 34 opened and flattened. In actual practice, the top and bottom ends of each of the springs are typically connected with each other, to form a complete ring. For some applications of the configuration described with reference to FIGS. 7A-B, stent-graft 20 comprises, in addition to tapered stent spring 40, a plurality of non-tapered stent springs 42, which may be disposed on both longitudinal sides of tapered stent spring 40 (and aorto-iliac bifurcation 102). Alternatively, all of the stent springs of stent-graft 20 are tapered (configuration not shown).

As used in the present application, including in the claims, “tubular” means having the form of an elongated hollow object that defines a conduit therethrough. A “tubular” structure may have varied cross-sections therealong, and the cross-sections are not necessarily circular. For example, one or more of the cross-sections may be generally circular, or generally elliptical but not circular.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
PCT Application PCT/IL2008/000287, filed Mar. 5, 2008, which published as PCT Publication WO 2008/107885 to Shalev et al., and U.S. application Ser. No. 12/529,936 in the national stage thereof, which published as US Patent Application Publication 2010/0063575
U.S. application Ser. No. 12/529,936, which published as US Patent Application Publication 2010/0063575 to Shalev et al.
U.S. Provisional Application 60/892,885, filed Mar. 5, 2007
U.S. Provisional Application 60/991,726, filed Dec. 2, 2007
U.S. Provisional Application 61/219,758, filed Jun. 23, 2009
U.S. Provisional Application 61/221,074, filed Jun. 28, 2009
PCT Application PCT/IB2010/052861, filed Jun. 23, 2010
PCT Application PCT/IL2010/000564, filed Jul. 14, 2010
PCT Application PCT/IL2010/000917, filed Nov. 4, 2010
PCT Application PCT/IL2010/000999, filed Nov. 30, 2010, entitled, “Multi-component stent-graft system for implantation in a blood vessel with multiple branches”
PCT Application PCT/IL2010/001018, filed Dec. 2, 2010, entitled, “Endovascular fenestrated stent-grafting”
PCT Application PCT/IL2010/001037, filed Dec. 8, 2010, entitled, “Endovascular stent-graft system with fenestrated and crossing stent-grafts”
a PCT application filed Feb. 8, 2010, entitled, “Thermal energy application for prevention and management of endoleaks in stent-grafts”

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising a stent-graft, which comprises:
a graft, which is shaped so as to define at least one generally tubular portion having a longitudinal axis, when the stent-graft is in a radially-expanded state; and
annular stent springs, which include at least first, second, and third tapered stent springs, which tapered stent springs are coupled to the portion of the graft, and each of which tapered stent springs comprises stent cells that taper to a set of one or more circumferentially-adjacent narrowest stent cells within the spring, such that the tapered stent springs comprise respective narrowest stent cell sets, when the stent-graft is in its radially-expanded state, wherein the narrowest stent cells within the spring are those stent cells within the spring that have the smallest lateral width, measured longitudinally in a direction parallel to the longitudinal axis,
wherein the first and the second tapered stent springs are axially adjacent, and the second and the third tapered stent springs are axially adjacent, and
wherein the narrowest stent cell sets of the first and the second tapered stent springs are rotationally positioned on the portion of the graft with a first non-zero relative angle shift between respective circumferential centers of the narrowest stent cell sets thereof, and the narrowest stent cell sets of the second and the third tapered stent springs are rotationally positioned on the portion of the graft with a second non-zero relative angle shift between respective circumferential centers of the narrowest stent cell sets thereof.

2. The apparatus according to claim 1, wherein the first and the second relative angle shifts are equal.

3. The apparatus according to claim 1, wherein the first and the second tapered stent springs do not touch one another when the stent-graft is straight in its radially-expanded state.

4. The apparatus according to claim 1, wherein each of the narrowest stent cell sets comprises exactly one of the stent cells.

5. The apparatus according to claim 1, wherein each of the first and the second relative angle shifts is between 120 and 150 degrees.

6. The apparatus according to claim 1, wherein each of the first and the second relative angle shifts is between 30 and 120 degrees.

7. The apparatus according to claim 1, wherein each of the first and the second relative angle shifts is less than 90 degrees.

8. The apparatus according to claim 1, wherein each of the first and the second relative angle shifts is greater than 5 degrees.

9. The apparatus according to claim 1, wherein the portion of the graft is disposed inside the tapered stent springs.

10. The apparatus according to claim 1, wherein the portion of the graft is disposed outside the tapered stent springs.

11. The apparatus according to claim 1, wherein at least a portion of the stent cells are closed.

12. The apparatus according to claim 11, wherein at least a portion of the stent cells are diamond-shaped.

13. The apparatus according to claim 1, wherein at least a portion of the stent cells are open.

14. The apparatus according to claim 13, wherein at least a portion of the stent cells are serpentine-shaped.

15. The apparatus according to claim 14, wherein at least a portion of the stent cells are zigzagged.

16. The apparatus according to claim 1, wherein a portion of the stent cells of one of the tapered stent springs are open, and one of the stent cells of the one of the tapered stent springs is closed.

17. The apparatus according to claim 16, wherein the portion of the graft is shaped so as to define a side-facing fenestration surrounded by the closed one of the stent cells.

18. The apparatus according to claim 16, wherein the closed one of the stent cells is at least as large as the other stent cells of the one of the tapered stent springs.

19. The apparatus according to claim 1, wherein the stent-graft includes a bifurcated portion.

20. Apparatus comprising a stent-graft, which comprises:
a graft, which is shaped so as to define at least one generally tubular portion when the stent-graft is in a radially-expanded state; and
annular stent springs, which include at least first, second, and third tapered stent springs, which tapered stent springs are coupled to the portion of the graft, and each of which tapered stent springs comprises stent cells that taper to a set of one or more circumferentially-adjacent smallest stent cells within the spring, such that the tapered stent springs comprise respective smallest stent cell sets, when the stent-graft is in its radially-expanded state,
wherein the first and the second tapered stent springs are axially adjacent, and the second and the third tapered stent springs are axially adjacent,
wherein the smallest stent cell sets of the first and the second tapered stent springs are rotationally positioned on the portion of the graft with a first non-zero relative angle shift between respective circumferential centers of the smallest stent cell sets thereof, and the smallest stent cell sets of the second and the third tapered stent springs are rotationally positioned on the portion of the graft with a second non-zero relative angle shift between respective circumferential centers of the smallest stent cell sets thereof, and
wherein each of the first and the second relative angle shifts is greater than 5 degrees.

21. The apparatus according to claim 20, wherein the first and the second relative angle shifts are equal.

22. The apparatus according to claim 20, wherein each of the smallest stent cell sets comprises exactly one of the stent cells.

23. The apparatus according to claim 20, wherein at least a portion of the stent cells are selected from the group consisting of: closed stent cells, and open stent cells.
